(19) United States
(12) Patent Application Publication
(10) Pub. No.: US 2004/0229954 A1
(43) Pub. Date: Nov. 18, 2004

(54) Selective manipulation of triglyceride, HDL and LDL parameters with 6-(5-carboxy-5-methyl-hexyloxy)-2,2-dimethylhexanoic acid monocalcium salt

(76) Inventors: Diane Elaine MacDougall, Ann Arbor, MI (US); Rebecca Guggemos Bakker-Arkema, Ann Arbor, MI (US)

Correspondence Address: WARNER-LAMBERT COMPANY, 2800 PLYMOUTH RD, ANN ARBOR, MI, 48105, US

(21) Appl. No.: 10/844,879

(22) Filed: May 13, 2004

Related U.S. Application Data
(60) Provisional application No. 60/471,147, filed on May 16, 2003.

(51) Int. Cl. A61K 31/19

(52) U.S. Cl. 514/574

(57) ABSTRACT

Methods for selectively modulating triglyceride, high-density lipoprotein, and low-density lipoprotein levels in a patient by administering a certain dose of 6-(5-carboxy-5-methyl-hexyloxy)-2,2-dimethylhexanoic acid monocalcium salt are disclosed.

### CROSS REFERENCE TO RELATED APPLICATION

This application claims priority of U.S. Provisional Application No. 60/471,147, filed May 16, 2003.

### FIELD OF THE INVENTION

This invention concerns a method for selectively modulating triglyceride, high-density lipoprotein, and low-density lipoprotein levels in a patient by administering a certain dose of 6-(5-carboxy-5-methyl-hexyloxy)-2,2-dimethylhexanoic acid monocalcium salt.

### BACKGROUND OF THE INVENTION

There are several forms of circulating blood cholesterol which occur naturally in mammals. Some forms are considered “bad” cholesterol, while other forms are considered “good” cholesterol and are essential for good health. The good form of cholesterol has been established to be HDL. It is widely believed that HDL is a “protective” lipoprotein and that increasing plasma levels of HDL may offer a direct protection against the development of cardiovascular disease. Numerous studies have demonstrated that both the risk of coronary heart disease (CHD) in humans and the severity of experimental atherosclerosis in animals are inversely correlated with serum HDL cholesterol (HDL-C) concentrations.

A bad cholesterol is LDL. Several clinical studies have established that lowering LDL-cholesterol in a mammal is an effective way to treat and prevent heart attacks, sudden death, and angina, both in subjects having higher than normal levels of circulating cholesterol, as well as those having normal levels of cholesterol. Lowering LDL is now one of the primary objectives of physicians treating patients who have, or who have a high risk of developing, cardiovascular diseases such as coronary heart disease, atherosclerosis, myocardial infarction, stroke, cerebral infarction, and even restenosis following balloon angioplasty. Many physicians are now utilizing cholesterol lowering agents purely as a prophylactic treatment in healthy subjects whose cholesterol levels are normal, thereby guarding against development of cardiovascular diseases.

The link between high levels of triglycerides (TG) and cardiovascular disease is well established. High blood levels of triglycerides, along with or independently of high blood levels of blood cholesterol, contributes to cardiovascular disease which is often manifested by chronic high blood pressure and increased risk of incapacitating and often fatal coronary attacks, stroke, etc. Triglyceride lowering is recognized as a desirable therapeutic goal given that elevated triglyceride levels are positively associated with pancreatitis and coronary artery disease in humans and are commonly seen in Type IV and Type V hyperlipoproteinemic patients and are associated with obesity, diabetes, and chronic renal failure.

Because vascular diseases such as coronary heart disease, atherosclerosis, stroke, and even peripheral vascular disease, remain a leading cause of death and disability throughout the world, the need continues to develop new and improved treatments, as well as agents that will actually prevent the formation of these diseases.

We have now discovered that treatment and prevention of cardiovascular diseases through the modification of triglyceride, high-density lipoprotein, and low-density lipoprotein levels in a patient can be effected by administering certain doses of 6-(5-carboxy-5-methyl-hexyloxy)-2,2-dimethylhexanoic acid monocalcium salt.

### SUMMARY OF THE INVENTION

Generally, the present invention relates to methods for modifying triglyceride, high-density lipoprotein, and low-density lipoprotein levels in a patient by administering certain doses of 6-(5-carboxy-5-methyl-hexyloxy)-2,2-dimethylhexanoic acid monocalcium salt, also known as gemcabene. One embodiment is a method of lowering serum triglyceride levels in a patient with elevated serum triglyceride levels including administering about 300 mg or less of 6-(5-carboxy-5-methyl-hexyloxy)-2,2-dimethylhexanoic acid monocalcium salt.

Another embodiment is a method of raising high density lipoprotein-cholesterol levels in a patient with elevated serum triglyceride levels including administering 300 mg or less of 6-(5-carboxy-5-methyl-hexyloxy)-2,2-dimethylhexanoic acid monocalcium salt.

Another embodiment is a method of lowering serum triglyceride levels and raising high density lipoprotein-cholesterol levels in a patient with elevated serum triglyceride levels comprising administering 300 mg or less of 6-(5-carboxy-5-methyl-hexyloxy)-2,2-dimethylhexanoic acid monocalcium salt.

Another embodiment is a method of lowering low density lipoprotein-cholesterol levels in a patient including administering greater than 300 mg of 6-(5-carboxy-5-methyl-hexyloxy)-2,2-dimethylhexanoic acid monocalcium salt.

Another embodiment is a method of lowering serum triglyceride levels without lowering low density lipoprotein-cholesterol levels in a patient with elevated serum triglyceride levels including administering 300 mg or less of 6-(5-carboxy-5-methyl-hexyloxy)-2,2-dimethylhexanoic acid monocalcium salt.

Another embodiment is a method of raising high density lipoprotein-cholesterol levels without lowering low density lipoprotein-cholesterol levels in a patient with elevated serum triglyceride levels including administering 300 mg or less of 6-(5-carboxy-5-methyl-hexyloxy)-2,2-dimethylhexanoic acid monocalcium salt.

Another embodiment is a method of lowering serum triglyceride levels and raising high density lipoprotein-cholesterol levels without lowering low density lipoprotein-cholesterol levels in a patient with elevated serum triglyceride levels including administering 300 mg or less of 6-(5-carboxy-5-methyl-hexyloxy)-2,2-dimethylhexanoic acid monocalcium salt.

Yet another embodiment is a method of lowering low density lipoprotein-cholesterol levels without lowering serum triglyceride levels or raising high density lipoprotein-cholesterol levels in a patient comprising administering greater than 300 mg of 6-(5-carboxy-5-methyl-hexyloxy)-2,2-dimethylhexanoic acid monocalcium salt.

### DETAILED DESCRIPTION OF THE INVENTION

The present invention provides methods for selectively modulating triglyceride, high-density lipoprotein, and low-density lipoprotein levels in a patient by administering a certain dose of 6-(5-carboxy-5-methyl-hexyloxy)-2,2-dimethylhexanoic acid monocalcium salt. 6-(5-Carboxy-5-methyl-hexyloxy)-2,2-dimethylhexanoic acid monocalcium salt, as well as its various crystal forms can be utilized in the presently claimed methods, are known and can be readily prepared as described in U.S. Pat. No. 5,648,387 and U.S. patent application Ser. No. 10/018,617 which are hereby expressly incorporated by reference.

While the present invention is not so limited, an appreciation of various aspects of the invention will be gained through the following discussion and the examples provided below.

All of the compounds to be utilized are either known or are readily prepared as described by Wetterau et al., U.S. Pat. No. 5,595,872 which is hereby expressly incorporated by reference.

As used in this specification and the appended claims, the singular forms “a”, “an”, and “the” include plural referents unless the content clearly dictates otherwise.

The recitation of numerical ranges by endpoints includes all numbers subsumed within that range (e.g. 1 to 5 includes 1, 1.5, 2, 2.75, 3, 3.80, 4, and 5).

The term “patient” as used herein includes all mammals including humans. Examples of patients include humans, cows, dogs, cats, goats, sheep, pigs, and rabbits.

The term “about” as used herein applies to all numeric values, whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The term “elevated serum triglyceride levels” as used herein refers to triglyceride levels greater than about 150 mg/dL. Typical elevated serum triglyceride levels are greater than, or equal to, 200 mg/dL.

The term “low HDL levels” as used herein refers to high density lipoprotein levels less than about 35 mg/dL.

A study was conducted which evaluated the efficacy and tolerability of 6-(5-carboxy-5-methyl-hexyloxy)-2,2-dimethylhexanoic acid monocalcium salt. The results of that study prompted the unexpected discovery that 6-(5-carboxy-5-methyl-hexyloxy)-2,2-dimethylhexanoic acid monocalcium salt, when administered to a patient at a particular dosage level, has selective LDL, or HDL and triglyceride modulating effects on the patient. This unexpected selective LDL, or HDL and triglyceride modulating effect has major health implications.

The present invention provides for methods of selectively modulating LDL, or HDL and triglyceride levels in a patient. One example of a method of the present invention is a method of lowering serum triglyceride levels in a patient with elevated serum triglyceride levels including administering 300 mg or less of 6-(5-carboxy-5-methyl-hexyloxy)-2,2-dimethylhexanoic acid monocalcium salt.

Another example of a method of the present invention is a method of raising high density lipoprotein-cholesterol levels in a patient with elevated serum triglyceride levels including administering 300 mg or less of 6-(5-carboxy-5-methyl-hexyloxy)-2,2-dimethylhexanoic acid monocalcium salt.

Another example of a method of the present invention is a method of lowering serum triglyceride levels and raising high density lipoprotein-cholesterol levels in a patient with elevated serum triglyceride levels comprising administering 300 mg or less of 6-(5-carboxy-5-methyl-hexyloxy)-2,2-dimethylhexanoic acid monocalcium salt.

Another example of a method of the present invention is a method of lowering serum triglyceride levels without lowering low density lipoprotein-cholesterol levels in a patient with elevated serum triglyceride levels including administering 300 mg or less of 6-(5-carboxy-5-methyl-hexyloxy)-2,2-dimethylhexanoic acid monocalcium salt.

Another example of a method of the present invention is a method of raising high density lipoprotein-cholesterol levels without lowering low density lipoprotein-cholesterol levels in a patient with elevated serum triglyceride levels including administering 300 mg or less of 6-(5-carboxy-5-methyl-hexyloxy)-2,2-dimethylhexanoic acid monocalcium salt.

Another example of a method of the present invention is a method of lowering serum triglyceride levels and raising high density lipoprotein-cholesterol levels without lowering low density lipoprotein-cholesterol levels in a patient with elevated serum triglyceride levels including administering 300 mg or less of 6-(5-carboxy-5-methyl-hexyloxy)-2,2-dimethylhexanoic acid monocalcium salt.

Additional examples of any of the methods discussed above, include administering from about 150 mg to about 300 mg of 6-(5-carboxy-5-methyl-hexyloxy)-2,2-dimethylhexanoic acid monocalcium salt. Other additional examples of any of the methods discussed above, include administering 150 mg to 300 mg of 6-(5-carboxy-5-methyl-hexyloxy)-2,2-dimethylhexanoic acid monocalcium salt. Further examples of any of the methods discussed above include administering 150 mg or 300 mg of 6-(5-carboxy-5-methyl-hexyloxy)-2,2-dimethylhexanoic acid monocalcium salt.

Another example of a method of the present invention is a method of lowering low density lipoprotein-cholesterol levels in a patient including administering greater than 300 mg of 6-(5-carboxy-5-methyl-hexyloxy)-2,2-dimethylhexanoic acid monocalcium salt.

Yet another example of a method of the present invention is a method of lowering low density lipoprotein-cholesterol levels without lowering serum triglyceride levels or raising high density lipoprotein-cholesterol levels in a patient comprising administering greater than 300 mg of 6-(5-carboxy-5-methyl-hexyloxy)-2,2-dimethylhexanoic acid monocalcium salt.

Additional examples of any of the methods discussed immediately above, include administering about 600 mg to about 900 mg of 6-(5-carboxy-5-methyl-hexyloxy)-2,2-dimethylhexanoic acid monocalcium salt. Additional examples of any of the methods discussed immediately above, include administering 600 mg to 900 mg of 6-(5-carboxy-5-methyl-hexyloxy)-2,2-dimethylhexanoic acid monocalcium salt. Further examples of any of the methods discussed immediately above include administering 600 mg or 900 mg of 6-(5-carboxy-5-methyl-hexyloxy)-2,2-dimethylhexanoic acid monocalcium salt.

Any of the methods may not only be practiced in patients with elevated serum triglyceride levels but also in patients with elevated serum triglyceride levels and low HDL levels as well as patients with low HDL levels alone.

6-(5-Carboxy-5-methyl-hexyloxy)-2,2-dimethylhexanoic acid monocalcium salt may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form should be pharmaceutically acceptable and substantially non-toxic in the amounts employed. In addition, 6-(5-carboxy-5-methyl-hexyloxy)-2,2-dimethylhexanoic acid monocalcium salt may be incorporated into sustained-release preparations and devices.

6-(5-Carboxy-5-methyl-hexyloxy)-2,2-dimethylhexanoic acid monocalcium salt may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating 6-(5-carboxy-5-methyl-hexyloxy)-2,2-dimethylhexanoic acid monocalcium salt in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Generally, the concentration of 6-(5-carboxy-5-methyl-hexyloxy)-2,2-dimethylhexanoic acid monocalcium salt in a semi-solid or solid composition such as a gel or a powder will be about 0.1-5 wt-%, preferably about 0.5-2.5 wt-%.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations

### EXAMPLES

Study Design

A randomized, double-blind, placebo-controlled, parallel group, dose-response, multicenter study was conducted. The study was conducted at 11 centers in the United States and one center in Canada. Institutional Review Board approval was obtained at each center and every patient was informed of the study, freely consented to participate and signed an informed consent document. Following a 6-week, single-blind placebo, dietary lead in period conducted according to National Cholesterol Education Program Step 1 Diet, eligible patients with HDL-C level <35 mg/dL (0.9 mmol/L) were stratified according to whether mean serum TG levels, calculated from measures at 2 and 4 weeks prior to randomization, were <200 mg/dL (2.3 mmol/L) or >200 mg/dL (2.3 mmol/L). Within each TG stratum, patients were randomized to receive either 150, 300, 600, or 900 mg of gemcabene or placebo daily once daily for 12 weeks.

Patients

Eligible patients were women of non-childbearing potential (naturally postmenopausal or surgically sterilized) or men 18 to 80 years of age with a baseline serum HDL-C levels <35 mg/dL. Patients were excluded if they had creatine kinase serum levels >3 times the upper limit of normal, a body mass index >35 kilogram/meter², uncontrolled hypertension defined as sitting diastolic blood pressure >95 mm Hg whether taking or not taking an acceptable antihypertensive medication, uncontrolled diabetes mellitus (Hemoglobin A_{1C}>10%), hepatic dysfunction including asparate aminotransferase or alanine aminotransferase levels >2 times the upper limits of normal, renal dysfunction as defined by blood urea nitrogen or creatinine levels >2 times upper limits of normal or uncontrolled hypothyroidism (thyroid stimulating hormone >1.5 times the upper limits of normal). Also excluded were those with a prior history of known gall bladder disease or pancreatitis, a history of consuming >14 alcoholic drinks per week, or those with a known hypersensitivity to lipid-altering drugs. Patients who had myocardial infarction, severe or unstable angina pectoris, coronary artery bypass graft or any other cardiovascular event requiring hospitalization within the last 3 months were also excluded from the study. Patients were not permitted any other lipid-altering drugs during the course of the study and if on prestudy lipid-altering drug therapy, were required to undergo an additional 4-week washout period. Use of isotretinoin, insulin, warfarin, immunosuppressive agents and intermittent systemic steroids were also prohibited. Alpha-blockers, β-blockers, hormone replacement therapy and oral hypoglycemic agents were permitted provided that their use was stable for at least 3 months.

Efficacy Parameters

The primary efficacy parameter was percent change from baseline in serum HDL-C levels at the study subject's last visit. Other efficacy parameters included percent change from baseline in serum LDL-C, TG, apolipoproteins A-I, A-II, B, C-III, E, and non-HDL-C levels. Additionally, nuclear magnetic resonance (NMR) spectroscopy was used to assess VLDL, HDL and LDL particle sizes as well as the total concentration of LDL particles. For HDL-C, non-HDL-C, LDL-C, and TG levels, the baseline value was the mean plasma value of two measurements, one obtained 2 weeks prior to randomization and one obtained at randomization. For all other parameters, the baseline value was defined as the single measurement obtained at randomization.

Clinical and Laboratory Data

All blood samples were collected following a 12-hour fast and analyzed by Medical Research Laboratories in Highland Heights, Ky. Blood samples for routine chemistry and hematology profiles were measured at screening, randomization and 4, 8, and 12 weeks following the start of study medication. Basic lipid profiles (HDL-C, LDL-C, and TG) were measured at screening, 2 and 4 weeks prior to starting study medication (during the baseline phase), at randomization, and at weeks 2, 4, 8 and 12 of the treatment phase. Other efficacy parameters were measured at randomization and study completion. Serum concentrations of cholesterol and TG were measured using an enzymatic, colorimetric assay on the Hitachi 747 analyzer. The HDL-C samples were obtained from the supemate after precipitation of the non-HDL lipoproteins using heparin and manganese chloride. Concentration of LDL-C was measured by β quantification, using a preparative ultracentrifuge fraction density >1.006 kg/L-HDL-C, when TG was >400 mg/dL. The LDL-C level was calculated using the Freidewald formula when TG was ≦400 mg/dL.³ Concentrations of apolipoproteins A-I, A-II, B and E were measured using immunonephelometry on the Dade Behring nephelometer. Concentration of apolipoprotein C-III was measured by electroimmunodiffusion using Hydragel LP CIII kits (Serbia, Issy-les-Moulineaux, France). Non-HDL-C was calculated by subtracting the measured HDL-C from the measured total cholesterol. Patients were observed and queried in a nonspecific fashion at each visit during the study for any new or continuing symptoms. Adverse events were recorded at each clinic visit and up to 15 days following cessation of treatment.

Statistical Analyses

A sample size of 15 patients per treatment group was planned to provide >90% power to detect a 30% difference in the percent change in HDL-C from baseline to week 12 between the placebo group and at least one gemcabene dose group in each triglyceridemic stratum. This calculation assumed a Dunnett-adjusted 2-sided alpha of 0.05 and a common standard deviation of 18%.

Within each TG stratum, an analysis of covariance model with the effects of baseline lipid value, treatment, and site was used to analyze the percent change from baseline at the last visit for each of the efficacy parameters by producing least squares means and p-values. Dunnett's multiple comparison procedure was used to adjust the p-values in the analysis of the primary endpoint of percent change in HDL-C. All other endpoint p-values were unadjusted for multiplicity.

The Shapiro-Wilk Test for Normality and visual analysis of the residuals was used to determine if the assumption of normality was reasonable. Since LDL-C levels in patients with TG≧2200 mg/dL, apolipoprotein A-I levels in patients with TG <200 mg/dL, and TG, apolipoproteins B and E levels in all patients were not normally distributed, median percent changes are presented and Conover's nonparametric Analysis of Covariance was used to analyze the ranked data for these parameters.

Baseline Demographics

A total of 161 patients were randomized. Of these patients, 67 had TG levels <200 mg/dL (14 randomized to placebo and 53 to active treatment) and 94 had TG levels ≧200 mg/dL (18 randomized to placebo and 76 to active treatment). Patient characteristics were generally similar across the TG strata (Table 1) with the obvious exception of the lipid parameters (Table 2). The study was completed by 152 patients. Six withdrew due to adverse events and 3 failed to complete the study for administrative or personal reasons. Compliance to study medication regimen was assessed at clinic visits by tablet count and found to be similar among the treatment groups. At the end of the study, 97% of placebo treatment and 96% of the active treatment patients were at least 80% compliant.

TABLE 1

| | Triglycerides <200 mg/dL | | | | | Triglycerides ≧200 mg/dL | | | | |
|---|---|---|---|---|---|---|---|---|---|---|
| | | gemcabene | | | | | gemcabene | | | |
| | Placebo | CI-1027 | CI1027 | CI-1027 | CI1027 | | CI-1027 | CI-1027 | CI-1027 | CI-1027 |
| | (N = | 150 mg | 300 mg | 600 mg | 900 mg | Placebo | 150 mg | 300 mg | 600 mg | 900 mg |
| Characteristic | 14) | (N = 14) | (N = 11) | (N = 14) | (N = 14) | (N = 18) | (N = 20) | (N = 21) | (N = 17) | (N = 18) |
| Men | 100% | 100% | 91% | 86% | 100% | 94% | 90% | 91% | 82% | 94% |
| Age, mean ± SE | 50 ± 3 | 55 ± 3 | 64 ± 3 | 51 ± 4 | 50 ± 3.0 | 53 ± 3 | 53 ± 2 | 54 ± 2 | 56 ± 3 | 58 ± 2 |
| Caucasian | 93% | 64% | 100% | 71% | 71% | 100% | 85% | 91% | 84% | 94% |
| Waist | | | | | | | | | | |
| Circumference | 100 ± 3 | 99 ± 3 | 108 ± 3 | 98 ± 4 | 100 ± 2 | 103 ± 3 | 103 ± 2 | 100 ± 3 | 98 ± 3 | 102 ± 2 |
| (cm), mean ± SE | | | | | | | | | | |
| Body Mass Index | 27 ± 1 | 29 ± 1 | 31 ± 1 | 28 ± 1 | 29 ± 1 | 30 ± 1 | 30 ± 1 | 28 ± 1 | 30 ± 1 | 29 ± 1 |
| (kg/m²), mean ± SE | | | | | | | | | | |
| Diabetes | 7% | 14% | 18% | 14% | 21% | 28% | 15% | 10% | 24% | 22% |
| Diastolic Blood | 77 ± 2 | 77 ± 2 | 75 ± 2 | 78 ± 2 | 76 ± 2 | 78 ± 2 | 79 ± 1 | 77 ± 2 | 80 ± 1 | 82 ± 2 |
| Pressure (mm Hg), | | | | | | | | | | |
| mean ± SE | | | | | | | | | | |
| Systolic Blood | 119 ± 5 | 119 ± 3 | 123 ± 3 | 116 ± 3 | 119 ± 4 | 122 ± 3 | 125 ± 3 | 122 ± 2 | 124 ± 3 | 132 ± 2 |
| Pressure (mm Hg), | | | | | | | | | | |
| mean ± SE | | | | | | | | | | |

TABLE 2

| | Triglycerides <200 mg/dL | | | | | Triglycerides ≧200 mg/dL | | | | |
|---|---|---|---|---|---|---|---|---|---|---|
| | | gemcabene | | | | | gemcabene | | | |
| | | CI-1027 | CI1027 | CI-1027 | CI1027 | | CI-1027 | CI-1027 | CI-1027 | CI-1027 |
| | Placebo | 150 mg | 300 mg | 600 mg | 900 mg | Placebo | 150 mg | 300 mg | 600 mg | 900 mg |
| Characteristic | (N = 14) | (N = 14) | (N = 11) | (N = 14) | (N = 14) | (N = 18) | (N = 20) | (N = 21) | (N = 17) | (N = 18) |
| LDL-C | 116 ± 10 | 107 ± 10 | 139 ± 11 | 108 ± 8 | 127 ± 9 | 101 ± 8 | 120 ± 9 | 108 ± 8 | 102 ± 9 | 110 ± 9 |
| (mg/dL) | | | | | | | | | | |
| NonHDL-C | 163 ± 11 | 154 ± 9 | 181 ± 11 | 141 ± 7 | 169 ± 10 | 183 ± 12 | 205 ± 9 | 201 ± 11 | 208 ± 16 | 201 ± 11 |
| (mg/dL) | | | | | | | | | | |
| Apo B (mg/dL) | 127 ± 7 | 114 ± 9 | 143 ± 10 | 113 ± 8 | 131 ± 7 | 130 ± 6 | 149 ± 7 | 147 ± 8 | 142 ± 8 | 131 ± 6 |
| HDL-C | 31 ± 1 | 32 ± 1 | 33 ± 1 | 33 ± 1 | 31 ± 1 | 29 ± 1 | 30 ± 1 | 28 ± 1 | 29 ± 1 | 29 ± 1 |
| (mg/dL) | | | | | | | | | | |
| TG (mg/dL) | 181 ± 12 | 170 ± 13 | 183 ± 1 | 151 ± 10 | 166 ± 17 | 367 ± 32 | 368 ± 40 | 428 ± 47 | 580 ± 133 | 382 ± 31 |
| Apo A1 | 114 ± 3 | 108 ± 4 | 112 ± 3 | 106 ± 4 | 105 ± 5 | 113 ± 3 | 116 ± 3 | 114 ± 4 | 118 ± 4 | 113 ± 3 |
| (mg/dL) | | | | | | | | | | |
| Apo A11 | 28 ± 1 | 28 ± 1 | 27 ± 1 | 27 ± 1 | 26 ± 1 | 28 ± 1 | 29 ± 1 | 30 ± 1 | 31 ± 2 | |
| 29 ± 1 | | | | | | | | | | |
| (mg/dL) | | | | | | | | | | |
| Apo CIII | 31 ± 2 | 28 ± 2 | 31 ± 2 | 26 ± 2 | 28 ± 3 | 56 ± 9 | 52 ± 5 | 61 ± 7 | 87 ± 21 | 62 ± 7 |
| (mg/dL) | | | | | | | | | | |
| Apo E (mg/dL) | 4.4 ± 0.3 | 4.2 ± 0.3 | 3.8 ± 0.3 | 3.8 ± 0.2 | 3.9 ± 0.2 | 6.4 ± 0.9 | 6.4 ± 0.6 | 7.1 ± 0.7 | 8.3 ± 1.1 | 6.8 ± 0.7 |

Efficacy Parameters

Percent change from baseline in HDL-C, LDL-C, TG and other efficacy parameters due to the administration of either 150, 300, 600 or 900 mg of gemcabene or placebo are shown in Table 3. In patients with mean baseline TG≧2200 mg/dL, gemcabene significantly increased serum HDL-C levels by 18% at the 150 mg dose with a corresponding increase of 6% in the levels of both apolipoprotein A-I and A-II and a decrease of 23% in apolipoprotein C-III. TG serum levels were significantly reduced by 27% and 39% at the 150 and 300 mg doses, respectively. No significant differences were found in serum HDL-C or TG levels at gemcabene doses of 600 or 900 mg versus placebo.

In patients with mean baseline TG <200 mg/dL, no significant differences were seen in serum HDL-C or TG levels in groups administered any dose of gemcabene versus placebo.

In both TG strata, gemcabene significantly reduced serum LDL-C levels by 15% to 25% at the 600 and 900 mg doses respectively, with corresponding decreases in the levels of apolipoprotein B. Also in both TG strata, gemcabene significantly reduced non-HDL-C levels by up to 18% at the higher doses.

TABLE 3
Percent Change from Baseline at Last Visit

| | LDL-C | HDL-C | TG |
|---|---|---|---|
| TG ≧200 mg/dL | | | |
| 150 mg | 16.4 (−26,255) | 17.6 ± 3.8* | −26.6** (−67, 46) |
| 300 mg | 8.3 (−31, 252) | 12.3 ± 3.8 | −38.9** (−73, 61) |
| 600 mg | −16.9* (−65, 386) | −0.9 ± 4.1 | −12.7 (−59, 38) |
| 900 mg | −23.0** (−72, 33) | −0.9 ± 3.8 | −9.0 (−56, |
| | | | 233) |
| Placebo | 0.4 (−24, 70) | 2.1 ± 4.0 | −4.7 (−64, 70) |
| TG <200 mg/dL | | | |
| 150 mg | −2.6 ± 4.4 | 6.2 ± 3.1 | −0.4 (−33, 32) |
| 300 mg | −5.6 ± 5.2 | 5.3 ± 3.5 | −14.6 (−55, 18) |
| 600 mg | −24.9 ± 4.6** | 0.5 ± 3.2 | 6.4 (−45, 97) |
| 900 mg | −15.2 ± 4.4** | 5.0 ± 3.2 | −8.1 (−35, 29) |
| Placebo | 1.6 ± 4.5 | 2.8 ± 3.2 | −8.3 (−24, |
| | | | 101) |
| | Apolipoprotein | Apolipoprotein | |
| | A-I | A-II | Apolipoprotein B |
| TG ≧200 mg/dL | | | |
| 150 mg | 5.8 ± (2.6)** | 5.9 ± 2.7* | 0.0 (−29, 33) |
| 300 mg | −3.1 ± (2.5) | 0.5 ± 2.6 | −6.5 (−30, 31) |
| 600 mg | −4.0 ± (2.8) | −0.8 ± 3.0 | −5.7 (−34, 54) |
| 900 mg | −2.7 ± (2.5) | −4.8 ± 2.6 | −9.2** (−33, 17) |
| Placebo | −4.6 ± (2.6) | −2.2 ± 2.8 | 6.2 (−21, 30) |
| TG <200 mg/dL | | | |
| 150 mg | 7.3 (−7, 40) | 5.5 ± 2.6* | −0.7 (−17, 13) |
| 300 mg | 1.3 (−11, 31) | 3.3 ± 2.8 | −11.7 (−33, 2) |
| 600 mg | 7.1 (−7, 89) | 7.2 ± 2.6* | −16.6* (−39, 77) |
| 900 mg | 5.5 (−11, 34) | 6.5 ± 2.5* | −15.7 (−38, 21) |
| Placebo | −2.1 (−19, 27) | −1.7 ± 2.6 | 0.6 (−22, 7) |
| | | Apolipoprotein | |
| | Apolipoprotein E | CIII | NonHDL-C |
| TG ≧200 mg/dL | | | |
| 150 mg | −20.0 (−52, 19) | −23.6 ± 5.7* | −1.6 ± 3.6 |
| 300 mg | −37.4** | −30.9 ± 5.6** | −9.4 ± 3.5 |
| | (−73, 117) | | |
| 600 mg | −25.4 (−49, 18) | −6.4 ± 6.5 | −10.9 ± 4.2 |
| 900 mg | −11.7 (−59, 65) | −10.8 ± 5.6 | −16.3 ± 4.3* |
| Placebo | 3.5 (−73, 102) | −5.4 ± 5.8 | −1.8 ± 3.8 |
| TG <200 mg/dL | | | |
| 150 mg | −7.7 (−42, 15) | −7.0 ± 9.2 | −2.5 ± 4.0 |
| 300 mg | −26.7** (−43, 63) | −14.3 ± 10.5 | −11.6 ± 4.2* |
| 600 mg | −13.7* (−38, 96) | 5.4 ± 9.0 | −17.6 ± 3.8** |
| 900 mg | −9.8* (−40, 11) | −9.5 ± 8.6 | −17.9 ± 3.6** |
| Placebo | −3.4 (−27, 44) | −5.4 ± 9.1 | 0.7 ± 3.7 |

Mean ± SE
For data not normally distributed, medians (min, max) are presented.
*p < 0.05 vs. placebo
**p < 0.01 vs. placebo

What is claimed is:

1. A method of lowering serum triglyceride levels in a patient with elevated serum triglyceride levels comprising administering about 300 mg or less of 6-(5-carboxy-5-methyl-hexyloxy)-2,2-dimethylhexanoic acid monocalcium salt.

2. The method of claim 1 comprising administering about 150 mg to about 300 mg of 6-(5-carboxy-5-methyl-hexyloxy)-2,2-dimethylhexanoic acid monocalcium salt.

3. The method of claim 1 comprising administering about 150 mg of 6-(5-carboxy-5-methyl-hexyloxy)-2,2-dimethylhexanoic acid monocalcium salt.

4. The method of claim 1 comprising administering about 300 mg of 6-(5-carboxy-5-methyl-hexyloxy)-2,2-dimethylhexanoic acid monocalcium salt.

5. A method of raising high density lipoprotein-cholesterol levels in a patient with elevated serum triglyceride levels comprising administering about 300 mg or less of 6-(5-carboxy-5-methyl-hexyloxy)-2,2-dimethylhexanoic acid monocalcium salt.

6. The method of claim 5 comprising administering about 150 mg to about 300 mg of 6-(5-carboxy-5-methyl-hexyloxy)-2,2-dimethylhexanoic acid monocalcium salt.

7. The method of claim 5 comprising administering about 150 mg of 6-(5-carboxy-5-methyl-hexyloxy)-2,2-dimethylhexanoic acid monocalcium salt.

8. The method of claim 5 comprising administering about 300 mg of 6-(5-carboxy-5-methyl-hexyloxy)-2,2-dimethylhexanoic acid monocalcium salt.

9. A method of lowering serum triglyceride levels and raising high density lipoprotein-cholesterol levels in a patient with elevated serum triglyceride levels (defined as those ≧200 mg/dL) comprising administering about 300 mg or less of 6-(5-carboxy-5-methyl-hexyloxy)-2,2-dimethylhexanoic acid monocalcium salt.

10. The method of claim 9 comprising administering about 150 mg to about 300 mg of 6-(5-carboxy-5-methyl-hexyloxy)-2,2-dimethylhexanoic acid monocalcium salt.

11. The method of claim 9 comprising administering about 150 mg of 6-(5-carboxy-5-methyl-hexyloxy)-2,2-dimethylhexanoic acid monocalcium salt.

12. The method of claim 9 comprising administering about 300 mg of 6-(5-carboxy-5-methyl-hexyloxy)-2,2-dimethylhexanoic acid monocalcium salt.

13. A method of lowering low density lipoprotein-cholesterol levels in a patient comprising administering greater than 300 mg of 6-(5-carboxy-5-methyl-hexyloxy)-2,2-dimethylhexanoic acid monocalcium salt.

14. The method of claim 13 comprising administering from about 600 mg to about 900 mg of 6-(5-carboxy-5-methyl-hexyloxy)-2,2-dimethylhexanoic acid monocalcium salt

15. The method of claim 13 comprising administering about 600 mg of 6-(5-carboxy-5-methyl-hexyloxy)-2,2-dimethylhexanoic acid monocalcium salt.

16. The method of claim 13 comprising administering about 900 mg of 6-(5-carboxy-5-methyl-hexyloxy)-2,2-dimethylhexanoic acid monocalcium salt.

17. A method of lowering serum triglyceride levels without lowering low density lipoprotein-cholesterol levels in a patient with elevated serum triglyceride levels (defined as those >200 mg/dL) comprising administering about 300 mg or less of 6-(5-carboxy-5-methyl-hexyloxy)-2,2-dimethylhexanoic acid monocalcium salt.

18. The method of claim 17 comprising administering about 150 mg to about 300 mg of 6-(5-carboxy-5-methyl-hexyloxy)-2,2-dimethylhexanoic acid monocalcium salt.

19. The method of claim 17 comprising administering about 150 mg of 6-(5-carboxy-5-methyl-hexyloxy)-2,2-dimethylhexanoic acid monocalcium salt.

20. The method of claim 17 comprising administering about 300 mg of 6-(5-carboxy-5-methyl-hexyloxy)-2,2-dimethylhexanoic acid monocalcium salt.

21. A method of raising high density lipoprotein-cholesterol levels without lowering low density lipoprotein-cholesterol levels in a patient with elevated serum triglyceride levels (defined as those ≧200 mg/dL) comprising administering about 300 mg or less of 6-(5-carboxy-5-methyl-hexyloxy)-2,2-dimethylhexanoic acid monocalcium salt.

22. The method of claim 21 comprising administering about 150 mg to about 300 mg of 6-(5-carboxy-5-methyl-hexyloxy)-2,2-dimethylhexanoic acid monocalcium salt.

23. The method of claim 21 comprising administering about 150 mg of 6-(5-carboxy-5-methyl-hexyloxy)-2,2-dimethylhexanoic acid monocalcium salt.

24. The method of claim 21 comprising administering about 300 mg of 6-(5-carboxy-5-methyl-hexyloxy)-2,2-dimethylhexanoic acid monocalcium salt.

25. A method of lowering serum triglyceride levels and raising high density lipoprotein-cholesterol levels without lowering low density lipoprotein-cholesterol levels in a patient with elevated serum triglyceride levels (defined as those ≧200 mg/dL) comprising administering about 300 mg or less of 6-(5-carboxy-5-methyl-hexyloxy)-2,2-dimethylhexanoic acid monocalcium salt.

26. The method of claim 25 comprising administering about 150 mg to about 300 mg of 6-(5-carboxy-5-methyl-hexyloxy)-2,2-dimethylhexanoic acid monocalcium salt.

27. The method of claim 25 comprising administering about 150 mg of 6-(5-carboxy-5-methyl-hexyloxy)-2,2-dimethylhexanoic acid monocalcium salt.

28. The method of claim 25 comprising administering about 300 mg of 6-(5-carboxy-5-methyl-hexyloxy)-2,2-dimethylhexanoic acid monocalcium salt.

29. A method of lowering low density lipoprotein-cholesterol levels without lowering serum triglyceride levels or raising high density lipoprotein-cholesterol levels in a patient comprising administering greater than 300 mg of 6-(5-carboxy-5-methyl-hexyloxy)-2,2-dimethylhexanoic acid monocalcium salt.

30. The method of claim 29 comprising administering from about 600 mg to about 900 mg of 6-(5-carboxy-5-methyl-hexyloxy)-2,2-dimethylhexanoic acid monocalcium salt.

31. The method of claim 29 comprising administering about 600 mg of 6-(5-carboxy-5-methyl-hexyloxy)-2,2-dimethylhexanoic acid monocalcium salt.

32. The method of claim 29 comprising administering about 900 mg of 6-(5-carboxy-5-methyl-hexyloxy)-2,2-dimethylhexanoic acid monocalcium salt.
